(19) United States
(12) Plant Patent Application Publication
(10) Pub. No.: US 2002/0170103 P1
(43) Pub. Date: Nov. 14, 2002

(54) Chrysanthemum plant named 95-157-6

(76) Inventors: Neil Owen Anderson, St. Paul, MN (US); Peter David Ascher, St. Paul, MN (US)

Correspondence Address: GARDNER, CARTON & DOUGLAS, PATENT DOCKET DEPT., 321 N. CLARK STREET - SUITE 3400, CHICAGO, IL, 60610, US

(21) Appl. No.: 09/503,380

(22) Filed: Feb. 14, 2000

(51) Int. Cl. A01H 5/00

(52) U.S. Cl. PLT/294

(57) ABSTRACT

A new and distinct Chrysanthemum plant named 95-157-6 is provided. This new cultivar was the result of a unique cross between Dendranthema weyrichii and Dendranthema grandiflora.

### BACKGROUND OF THE INVENTION

The present invention comprises a new and distinctive chrysanthemum plant, hereinafter referred to by the cultivar name 95-157-6. This new cultivar was the result of a cross in 1989 between Dendranthema weyrichii and Dendranthema grandiflora. More specifically, the breeding program which resulted in the production of the new cultivar was carried out at St. Paul, Minn. The female or seed parent of 95-157-6 was a Dendranthema weyrichii, commercially available from White Flower Farms, Connecticut having the following characteristics: (a) the plant habit is prostrate and the plant spreads via rhizomes to form a large mat after the first year; (b) the plant dimensions are that the plant has a diameter of about 1.5′ and is about 5-6″ tall; (c) the plant is hardy in zones 4-9 (Southeast)/Zone 10 (west); (d) the flower of the plant is a single daisy, having light lavender-colored ray florets and central disc florets with yellow pollen; (e) the plant has leaves that are dark green in color, with a very shiny leaf surface (glossy), and glabrous leaf margins that are deeply incised; and (d) the plant tends to rosette, needs cold treatment to flower consistently, flowering can be sporadic with gaps in the plant architecture and the plant is an obligate short-day plant. The male or pollen parent of 95-1576 was a Dendranthema grandiflora which is commercially available from Yoder Brothers, Inc., Barberton, Ohio having the following characteristics: (a) the plant habit is cushion; (b) the plant dimensions are that the plant is similar to other cushion types commercially available from Yoder Brothers, Inc., such as, but not limited to the variety, ‘Shasta’; (d) the plant is hardy in zones 6-9 (Southeast)/Zone 10 (west); (d) the flower is a single or duplex daisy, possibly orange or bronze ray florets, central disc florets with yellow pollen; (e) the plant has leaves that are similar to other Yoder Brothers, Inc. cushion series chrysanthemums; and (d) the plant is a facultative short-day plant. The resulting seeds, identified as 90-287-194 were collected. In 1991, a plant of 90-287-194 was crossed as the male parent with plants identified as 77-AM3-3, a University of Minnesota inbred parental selection, as the female parent and the resulting seeds, identified as cross number 92-279-2 were collected. In 1994, a plant of 92-279-2 was crossed as the male parent with plants of the cultivar ‘Baby Tears’, which is commercially available from Yoder Brothers, Inc., Barberton, Ohio, as the female parent and the resulting seeds, identified as cross number 95-157, were collected. In 1995, seedlings of the cross 95-157 were germinated and the flowering progeny evaluated. 95-157-6 was the sixth plant from the cross and was selected in the fall of 1995. The parentage of the new cultivar can be summarized as follows:

Dendranthema weytchii×Dendranthema grandiflora

Asexual reproduction of the new cultivar by terminal or stem cuttings taken during 1996 through 1999 at St. Paul, Minnesota, U.S.A. has demonstrated that the characteristics of the new cultivar as herein described are firmly fixed and are retained through successive generations of such asexual propagation.

### SUMMARY OF THE INVENTION

It was found that the cultivar of the present invention:

(a) exhibits extreme hybrid vigor,

(b) develops, in its second and subsequent years after planting, when grown in the fall under natural daylength and without the application of growth regulators, into a flowering herbaceous shrub having a plant height of from about 1.8 to about 2.25 feet and a spread from about 2.4 to about 5.0 feet,

(c) exhibits, in its second and subsequent years after planting and during the fall season (August-October), a massive floral display,

(d) displays flowers which are slightly toned with grey, giving the flower petals a slightly altered coloration,

(e) exhibits superior winter hardiness, including frost tolerance, and

(f) exhibits self-pinching.

The 95-157-6 cultivar has not been observed under all possible environmental conditions to date. Accordingly, it is possible that the phenotype may vary somewhat with variations in the environment, such as temperature, light intensity, and day length.

When the new cultivar of the present invention is compared to ‘Stephanie’ (U.S. Plant Pat. No. 9,445), it is found to exhibit a more spreading and prolific habit accompanied with a massive floral display in its second and subsequent years after planting. Reference is made to attached Chart A which compares certain characteristics of 95-157-6 to ‘Stephanie’.

### BRIEF DESCRIPTION OF THE PHOTOGRAPHS

The accompanying photographs show as nearly true as it is reasonably possible to make the same in color illustrations of this type, typical flower and foliage characteristics of the new cultivar. The plants were grown in a greenhouse at St. Paul, Minn., USA.

FIG. 1 shows an adaxial and abaxial views of the leaf shape of chrysanthemum variety 95-157-6.

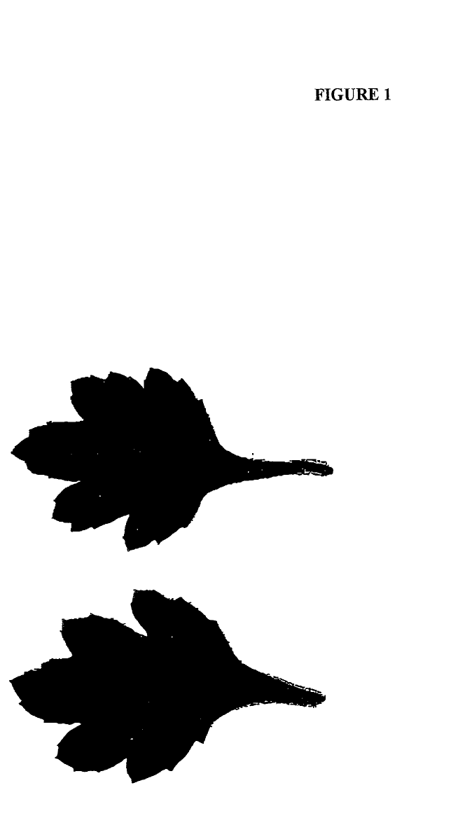

FIG. 2 shows the breeding history of chrysanthemum variety 95-157-6.

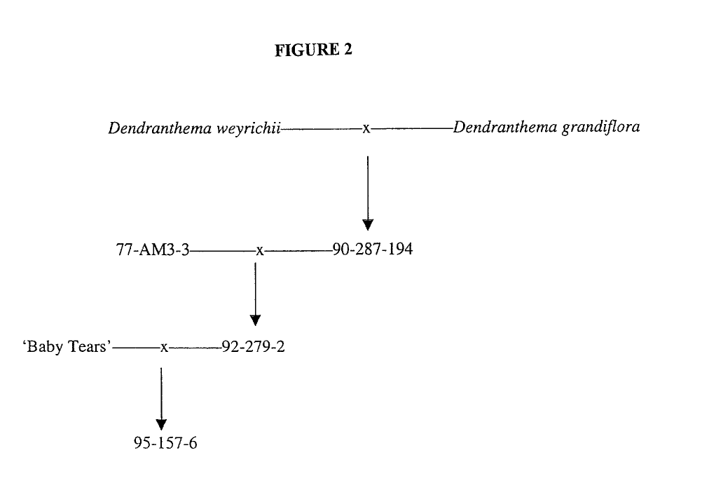

FIG. 3 is a color photograph of chrysanthemum variety 95-157-6 after one year of growth.

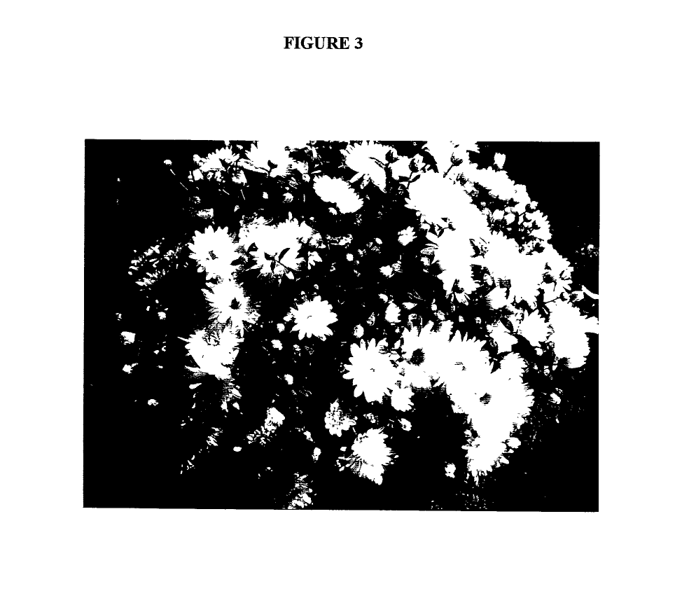

FIG. 4 is a color photograph of chrysanthemum variety 95-157-6 after two years of growth.

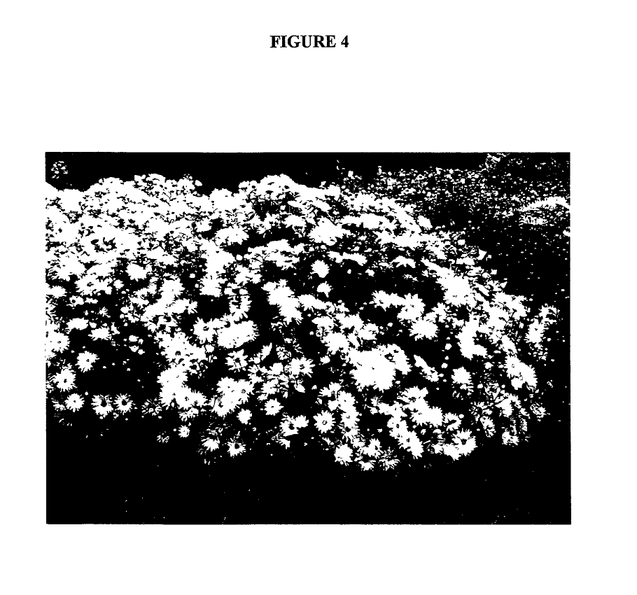

### DETAILED BOTANICAL DESCRIPTION

The chart used in the identification of colors described herein is the R.H.S. Colour Chart of The Royal Horticultural Society, London, England. The color values were determined on Oct. 15, 1999 in St. Paul, Minn. The readings were taken between 1:30 and 2:00 p.m. under approximately 2500 footeandles of light. The plants were produced from cuttings taken from stock plants and were grown under greenhouse conditions comparable to those used in commercial practice while utilizing a soilless growth medium and maintaining temperatures of approximately 72° F. during the day and approximately 65° F. during the night.

Propagation:

Type.—Herbaceous stem cutting

Time to rooting.—About 1 week

Rooting habit.—Vigorous

Botanical Classification: Dendranthema×hybrida

Commercial Classification: Chrysanthemum hybrid

Plant Description:

Appearance, shape.—Spherical mound

Appearance, growth habit.—Cushion

Appearance, growth rate/vigor.—Vigorous

Plant height.—about 18 to about 19 inches (first year) about 1.8 to about 2.25 feet (second year)

Lateral branch length.—1 to 2.5 feet

Quantity of lateral branches after of apical meristem.—removal One per node

Stem color.—RHS Paris Green 58/1

Foliage Description:

Number of leaves per plant.—Greater than 8,000 (second year)

Number of leaves per lateral branch.—5 to 20

Leaf arrangement.—Alternate

Leaf size, fully expanded, length.—7.8 cm

Leaf size, fully expanded, width.—4.8 cm

Leaf apex.—Mucronulate

Leaf base.—Cuneate

Leaf margin.—Incised (Mulberry-like incisions)

Leaf texture.—Mildly hirsute

Petiole length.—2.2 cm

Color, young foliage adaxial surface.—RHS Scheeles Green 860/2

Color, young foliage abaxial surface.—RHS Spinach Green o960/2

Color, fully expan ded foliage adaxial surface.—RHS Spinach Green o960

Color, fully expanded foliage abaxial surface.—RHS Spinach Green o960/1 to RHS Spinach Green o960/3

Color, venation adaxial surface.—RHS Spinach Green o960/2

Color, venation abaxial surface.—RHS Spinach Green o960/3

Color, petiole.—RHS Scheeles Green 860/1

Inflorescence Description:

Appearance.—Head (composite), pentaplex daisy

Flowering response.—About 6 weeks (SD)

Quantity of inflorescences.—About 1000 (first year) About 3,000 (second year)

Inflorescence size, diameter.—7.1 cm

Inflorescence size, depth (height).—3.4 cm

Inflorescence size, diameter of disc.—1.0 cm

Opening inflorescences, bud shape.—Dome shaped to upright tubular

Opening inflorescences, bud size, length.—0.6 cm

Opening inflorescences, bud size, width.—0.7 cm

Opening inflorescences, bud color.—RHS Creamy White

Ray florets, shape.—Linear lanceolate

Ray florets, size, length.—2.7 cm

Ray florets, size, width.—0.6 cm

Ray florets, apex.—Retuse

Ray florets, base.—Attenuate

Ray florets, margin.—Entire

Ray florets, texture.—Glabrous

Ray florets, aspect.—From about 45° vertical to slightly pendant 45°

Number of ray florets per inflorescence.—About 119

Ray florets, color, when opening, adaxial surface.—RHS Sap Green 62/3

Ray florets, color, when opening, abaxial surface.—RHS Uranium Green 63/3

Ray florets, color, mature, adaxial surface.—RHS Whit e

Ray florets, color, mature, abaxial surface.—RHS White

Ray florets, color, fading to.—RHS White

Disc florets, shape.—Tubular, rounded at tip

Disc florets, size, length.—0.7 cm

Disc florets, size, width.—0.2 cm

Number of disc florets per inflorescence.—About 100

Disc florets, color, immature.—RHS Straw Yellow 604

Disc florets, color, mature.—RHS Chinese Yellow 606

Peduncle, aspect, strength.—Stiff

Peduncle, aspect, angle to stem.—45°

Peduncle, length, first peduncle.—3 cm

Peduncle, length, fourth peduncle.—4.6 cm

Peduncle, texture.—Mildly hirsute

Peduncle, color.—RHS Viridian Green 55/3

Reproductive organs, androecium, floret location.—Disc florets

Anther color.—RHS Canary Yellow 2/1

Pollen, abundance.—Abundant

Pollen, color.—RHS Buttercup Yellow 5

Reproductive organs, gynoecium, floret location.—Disc/ray florets

Style color.—RHS Buttercup Yellow 5/2

Disease Resistance: None Known

Seed Production: About 219 ovules/flower

Winter Hardiness: Hardy in zones 3-10 in uncovered field conditions without the need for added protection such as snow fences, mulch, etc.

Frost Tolerance Yes, extends blooming season to the first freeze in the north (In zones 3-4 the first frost usually takes place between September 1-15. In zones 3-4, the first freeze usually takes place between October 1-20).

What is claimed is:

1. A new and distinct cultivar of Chrysanthemum named 95-157-6 substantially as herein shown and described, which:
(a) exhibits extreme hybrid vigor,
(b) develops, in its second and subsequent years after planting, when grown in the fall under natural daylength without the application of growth regulators, into a flowering herbaceous shrub having and without the application of growth regulators, into a flowering herbaceous shrub having a plant height of from about 1.8 to about 2.25 feet and a spread from about 2.4 to about 5.0 feet,
(c) exhibits, in its second and subsequent years after planting and during the fall season (August-October), a massive floral display,
(d) displays flowers which are slightly toned with grey, giving the flower petals a slightly altered coloration,
(e) exhibits superior winter hardiness, including frost tolerance, and
(f) exhibits self-pinching.

| CHARACTERISTIC | 95-157-6 | ‘Stephanie’ |
|---|---|---|
| Capitulum form and type | Pentaplex daisy | Flat daisy |
| Plant Height | About 18 to 19 inches (first | 10 to 12 inches |
| | year); about 1.8 to about 2.25 | |
| | feet (second year) | |
| Branching Pattern | Spreading and very prolific | Spreading and |
| | | prolific |
| Flowering Response | 6 weeks | 7 weeks |
| Flower diameter | 7.1 cm | 5.8 to 6.1 cm |
| Ray florets, color, mature | White | White |
